(19) United States
(12) Patent Application Publication
(10) Pub. No.: US 2013/0115638 A1
(43) Pub. Date: May 9, 2013

(54) METHOD AND RAPID TEST FOR DETERMINING THE FERTILITY OF SPERM

(71) Applicants: Klaus Steger, Giessen (DE); Agnieszka Paradowska, Linden (DE); Birte Bärmann, Giessen (DE)

(72) Inventors: Klaus Steger, Giessen (DE); Agnieszka Paradowska, Linden (DE); Birte Bärmann, Giessen (DE)

(73) Assignee: JUSTUS-LIEBIG-UNIVERSITAET GIESSEN, Giessen (DE)

(21) Appl. No.: 13/698,241

(22) PCT Filed: May 17, 2011
(86) PCT No.: PCT/EP11/57942

§ 371 Date: Jan. 25, 2013

(30) Foreign Application Priority Data
May 18, 2010 (EP) 10163147.1

(51) Int. Cl. G01N 33/569; G01N 33/53

(52) U.S. Cl. 435/7.93; 435/7.94; 435/7.1

(57) ABSTRACT

The present invention concerns a procedure and a device to determine the concentration of protamine-1 and protamine-2 and the ratio between protamine-1 and protamine-2 as protamine/protein ratio in a questionable sample from an individual to be examined in order to assess the fertility of the sperm in vitro. This assessment is of great importance for the prognosis of the success of an artificial insemination of an egg in vitro. The test strip comprises a sperm pretest to determine the sperm concentration and a protamine rapid test to determine the concentrations of protamine-1 and protamine-2 and the ratio between protamine-1 and protamine-2 as protamine/protein ratio.

The present invention relates to a procedure and a rapid test for determining the fertility of mammalian sperm, advantageously of human sperm. The fertility of sperm is determined by detection of proteins protamine-1 and protamine-2 and representation of the ratio between protamin-1 and protamin-2 (protamine/protein ratio). The protamine/protein ratio correlates with the fertility sperm. Procedure and rapid test are utilized to obtain a prognosis for the success of artificial insemination of an egg.

### Abbreviations and Definitions

DNA: Deoxyribonucleic acid
ELISA: Enzyme-linked immunosorbent assay

### Questionable Sample: \

A questionable sample is biological material of a male proband suitable to be used for a determination of sperm fertility. The questionable sample is notably ejaculate, testicular tissue or epididymis tissue containing sperm or spermatids. The determination of fertility is based on the detection of proteins protamine-1 and protamine-2 in the questionable sample and a representation of the ratio between protamine-1 and protamine-2 (protamine/protein ratio) which correlates with sperm fertility.

### Histones:

Proteins in the cell nucleus of eukaryotes which are part of the chromatin and thus play an important role in the packing of DNA.

### ICSI:

Intracytoplasmic sperm injection; procedure for artificial insemination of an egg.

### IU:

International unit

### IVF:

In-vitro fertilization; procedure for artificial insemination of an egg.

mL:

Milliliter

mM:

Millimole

mRNA:

Messenger RNA, RNA-transcript of a specific DNA section which represents a gene.

### Nucleic Acid Sequence:

Sequence of nucleotides of any nucleic acid, for example DNA or RNA.

### P-1:

Protein protamine-1

### P-2:

Protein protamine-2

### PCR:

Polymerase chain reaction; procedure for in vitro amplification of nucleic acid sequences.

### Protamine:

Arginine-rich proteins in the nucleus of mammalian (including human) sperm cells which replace histones during the late haploid stage of spermatogenesis.

### Protamine/mRNA Ratio:

Ratio between the amount of protamine-1-specific mRNA and the amount of protamine-2-specific mRNA in a questionable sample.

### Protamine/Protein Ratio:

Ratio between the amount of protamine-1 and the amount of protamine-2 in a questionable sample. A protamine/protein ratio of 1:1 is generally considered as indicative of positive fertility of sperm or spermatids in the questionable sample. According to this invention, also values within a protamine/protein ratio threshold range of 0.8:1.2 are still considered as indicative of positive fertility of sperm or spermatids in the questionable sample.

### RNA:

Ribonucleic acid

rpm:

Rounds per minute

### Spermatogenesis:

Multiplication and differentiation of male germ cells in the testicles resulting in the formation of sperm.

### Spermatids:

Sperm precursor cells

### TESE:

Testicular sperm extraction: removal of testicular tissue in order to gather spermatids or sperm.

### DESCRIPTION OF THE GENERAL SECTION OF INVENTION

Fertile sperm is a prerequisite to fertilize an egg. Since insufficient sperm fertility may play a crucial role in the case of unfulfilled desire for children, the diagnosis of sperm fertility is of great importance in human and veterinary medicine. Particularly when procedures for artificial insemination of eggs are applied, a prior determination of the fertility of utilized sperm is essential.

During spermatogenesis, DNA-binding histones are replaced by protamines. Two different protamines are present in humans, referred to as protamine-1 (P-1) and protamine-2 (P-2). A detection of protamines can be performed in testicular tissue samples or the ejaculate.

The qualitative detection of protamines in testicular tissue samples is indicative of the presence of sperm and serves as predictive factor for a testicular sperm extraction (TESE).

The quantitative detection of protamines in testicular tissue samples or ejaculates provides information with respect to the fertilization potential of the present sperm. Surprisingly, in particular the ratio of protamine-1 to protamine-2 (for example determined by indirect means as protamine/mRNA ratio) represents a valid prognostic marker for the success of a subsequent in-vitro fertilization or intracytoplasmic sperm injection. The detection of protamines for determining the fertility of sperm is specific, since protamines are only found in male haploid gametes (and thus also in sperm). For a determination of the fertility of sperm, in particular of human sperm, various methods are known in the state of the art.

### State of the Art

Prior art knows in particular the ejaculate analysis for a determination of sperm fertility, e.g. in accordance with WHO criteria. In this procedure, amongst others the absolute sperm count per volume unit is determined and sperm morphology and motility are assessed. Disadvantageous however is that no prognostic factors for the success of artificial insemination can be derived from ejaculate analyses. Specific laboratory equipment is required to conduct this procedure which can only be performed by trained expert personnel.

A further procedure is the histological examination of a testicular tissue sample in which the presence and morphology of sperm in testicular tissue is assessed. Disadvantageous is however that these histological examinations do not allow a statement whether the existing sperm is functionally intact, which is often not the case even though this could be assumed based on the morphology of the sperm. A reliable assessment of the fertilization potential of the existing sperm or a prognosis with respect to the success of artificial insemination of an egg is not possible with this procedure. The evaluation of sperm morphology is in addition strongly dependent on the experience of the examiner and can consequently not be standardized. Performing the procedure requires specific laboratory equipment as well as trained expert personnel and cannot be conducted by anyone or anywhere.

Another approach to determine the fertility of sperm was disclosed by the inventors themselves (Steger et al., 2007, Human Reproduction). This procedure is based on the detection of mRNA, in particular the detection of protamine-1 mRNA and the detection of protamine-2 mRNA, which are then put into relation with each other and result in the so-called protamine/mRNA ratio. The protamine/mRNA ratio provides information with respect to the fertility of the sperm, but this procedure is also associated with major disadvantages. This highly specific laboratory method requires, due to the sensitive nature of RNA and the fast degradation thereof, a particularly high degree of purity during isolation and handling and can only be carried out in reproducible quality by very well-trained expert personnel and with special PCR equipment. The procedure is highly error-prone, in addition comparatively time-consuming and expensive and altogether unsuitable as routine method to be carried out by anyone or anywhere.

### Aim

Aim of the present invention is therefore to provide a fast, standardizable and cost-efficient procedure for a reliable determination of the fertility of sperm which can be performed by anyone and anywhere without the need for particular skills or special laboratory equipment, as well as a rapid test to perform said procedure.

### Solution

The aim is solved according to the present invention by an immunological procedure in which the protamine/protein ratio is determined and by a rapid test to conduct this procedure.

Surprisingly it was diagnosed and medically confirmed that in sperm or spermatids samples of healthy men, a protamine/protein ratio which is the quantitative relation between protamine-1 and protamine-2 of 1:1 indicates normal fertility of the sperm.

This also applies to a certain tolerance range if sperm or spermatid samples show a protamine/protein ratio of 0.8-1.2 protamine-1:0.8-1.2 protamine-2 ratio instead of 1:1. If however the protamine/protein ratio in sperm or spermatid samples deviates from the above mentioned range, is this indicative of reduced fertility up to infertility of the sperm or spermatids in the questionable sample. This sperm is consequently not used for a subsequent in-vitro fertilization or intracytoplasmic sperm injection.

Numerous experimental analyses of the inventors with different human samples in question documented that the protamine/protein ratio which is the quantitative relation between protamine-1 and protamine-2 represents a valid prognostic marker for the success of subsequent in-vitro fertilization or intracytoplasmic sperm injection.

According to this invention, the determination of the protamine/protein ratio in sperm or spermatids is performed in a specific procedure and using ELISA or rapid test.

For this purpose, a questionable sample is processed in a suitable manner to isolate proteins and in particular protamine-1 and protamine-2 from the sperm or spermatids such that these are freely accessible and can be detected separately by immunological means. Subsequently, protamine-1 and protamine-2 are detected qualitatively and quantitatively and the protamine/protein ratio is determined, in particular by means of ELISA or in a rapid test.

In order to perform an ELISA, the processed sample is in a first assay brought into contact with an antibody specifically directed against protamine-1 (anti-protamine-1 antibody) and in a second assay brought into contact with an antibody specifically directed against protamine-2 (anti-protamine-2 antibody). If protamine-1 and/or protamine-2 is present in the questionable sample, binding between protamine-1 and the corresponding anti-protamine-1 antibody and/or between protamine-2 and the corresponding anti-protamine-2 antibody will occur. This binding can be visualized and quantified via a color reaction by contact with a secondary antibody whose epitope specifically binds both anti-protamine-1 antibody and anti-protamine-2 antibody and using suitable devices.

The binding of the secondary antibody is visualized through an enzymatic or physical color reaction.

The intensity of the color reaction is measured in protamine-1- and protamine-2 assays and values are then put into relation with one another, resulting in the protamine/protein ratio (ratio of the amount of protamine-1 to the amount of protamine-2). This protamine/protein ratio is a valid prognostic marker for the success of a subsequent in-vitro fertilization or intracytoplasmic sperm injection. If the protamine/protein ratio in sperm or spermatids in a questionable sample is in a range of 0.8-1.2 protamine-1:0.8-1.2 protamine-2, preferably equals 1:1, a normal sperm fertility exists and the sperm can be used for a subsequent in-vitro fertilization or intracytoplasmic sperm injection. If however the ratio deviates from the above mentioned range, is this indicative of reduced fertility up to infertility of the sperm or spermatids in the questionable sample. This sperm is not used for a subsequent in-vitro fertilization or intracytoplasmic sperm injection.

This procedure for the first time allows a fast, reliable, standardizable and cost-efficient determination of sperm fertility and thus provides a considerable improvement in determining the fertility of sperm and spermatids in a questionable sample.

Processing a questionable sample includes the following steps:
Extraction and concentration of sperm from a questionable sample;
Decondensation of the tight association of DNA, histones and protamines in the sperm to allow a release of proteins, in particular of protamine-1 and/or protamine-2;

### Cell Lysis

An extraction and concentration of sperm from a questionable sample like e.g. ejaculate, epididymis tissue or testicular tissue is preferably performed e.g. by sedimentation or by centrifugation.

Decondensation of the tight association of DNA, histones and protamines in the sperm of a questionable sample is achieved e.g. by incubation in a suitable buffer containing e.g. detergents or enzymes to release proteins, in particular proteins protamine-1 and/or protamine-2.

Alternatively, the total protein content of decondensed sperm in a sample is determined in order to be able to adjust a specific protein concentration, for example by methods known to the expert in this field like e.g. in a Bradford, Biuret or Lowry assay.

The exemplarily embodiments described herein are particularly advantageous embodiments but restrict by no means the content of the teaching of the present invention.

### EMBODIMENTS

### 1. Processing of a Questionable Sample

The extraction of sperm from a questionable sample like e.g. an ejaculate sample is carried out e.g. by centrifugation of the total ejaculate volume (which varies in humans between 0.5 and 6 mL) at 2 000 to 5 000 rpm in a suitable vial. The resulting sperm is thus concentrated in the form of a pellet.

Decondensation of the tight association of DNA, histones and protamines in the sperm is e.g. achieved by resuspending the sperm pellet in decondensation buffer e.g. 25 mM DTT (1,4-dithiothreitol; company Roche), 0.2% Triton X-100 (company Sigma), 200 IU heparin/mL (heparin sodium 5000 units, Ratiopharm) in PBS (phosphate buffered saline) e.g. with 2 mL buffer. Incubation is continued until the sperm heads have already swollen extensively, but the sperm core is still intact, which is monitored e.g. by microscopic observation. Incubation is advantageously continued for 5 to 30 min (depending on the respective ejaculate sample). The suspension containing decondensed sperm is then washed e.g. by addition of washing buffer (e.g. PBS-buffer with protease inhibitor) preferably up to twice.

Advantageously, 100 to 1000 μl washing buffer are used for this purpose and subjected to centrifugation each time until the sperm is sedimented, e.g. 5 to 15 min at 500 to 4 000 rpm. The sperm pellet is subsequently resuspended in 100 to 1000 μl washing buffer.

The following cell lysis is carried out e.g. by freeze-and-thaw cycles. The suspension containing decondensed sperm is treated e.g. for 5 to 20 min in an ultrasonic bath and subsequently on ice for 15 to 60 sec using a sonifier at 20 to 60% power.

The total protein content of the suspension containing decondensed sperm is measured e.g. according to a method known by the expert, e.g. in a Bradford, Biuret or Lowry assay.

The suspension with decondensed sperm is then adjusted to a suitable protein concentration of e.g. 0.1 μg/mL, 0.25 μg/mL, 0.5 μg/mL, 1.0 μg/mL, 2.5 μg/mL, 5.0 μg/mL, 10.0 μg/mL and/or 20 μg/mL with dilution buffer (e.g. PBS buffer with protease inhibitor).

In order to conduct the rapid test procedure, the questionable sample is processed by incubation of the ejaculate for liquefaction for 15 to 60 min at room temperature, addition of decondensation buffer and incubation for 5 to 30 min to obtain a suspension with decondensed sperm. Cell lysis is performed by addition of a lysis buffer e.g. 20 mM Tris-HCl (pH 7.5, 150 mM NaCl, 1 mM Na₂EDTA, 1 mM EGTA, 1% Triton, 2.5 mM sodium pyrophosphate, 1 mM beta-glycerophosphate, 1 mM Na₃VO₄, 1 μg/mL leupeptin).

Alternatively, an untreated questionable sample, e.g. ejaculate, is used.

The upper limit of protamine concentration in the rapid test is chosen such that even ejaculate samples with a sperm count of e.g. 200 million sperm/mL can be measured. The lower limit of protamine concentration was set to 0.2 million sperm/mL using a protamine-ELISA.

### 2. Measurement of Protamine-1 and Protamine-2 Concentrations in a Questionable Sample

### 2.1. ELISA-Procedure (Indirect Sandwich Procedure)

Starting material is the processed questionable sample according to embodiment 1, i.e. a suspension containing decondensed sperm with a protein concentration adjusted to e.g. 0.1 μg/mL, 0.25 μg/mL, 0.5 μg/mL, 1.0 μg/mL, 2.5 μg/mL, 5.0 μg/mL, 10.0 μg/mL and/or 20 μg/mL.

The indirect sandwich procedure is a standard enzyme immunoassay. For this purpose, proteins protamine-1 and protamine-2 of the processed questionable sample according to embodiment 1 are immobilized on a solid phase e.g. of a 96-well microtiter plate in such a way that the distribution of these proteins is as homogenous as possible. Then protamine-specific antibodies are added (anti-protamine-1 antibodies or anti-protamine-2 antibodies) which bind during the first incubation step to proteins protamine-1 and protamine-2. Two separate assays are performed in this case, whereby in the first assay anti-protamine-1 antibody which binds to protamine-1 is added to the questionable sample and in a second assay anti-protamine-2 antibody which binds to protamine-2 is added to the same questionable sample.

Wells are subsequently washed and enzyme-conjugated secondary antibody whose epitope specifically binds to anti-protamine-1 antibody and anti-protamine-2 antibody is added to the rinsed and empty wells. In this second incubation step, the secondary antibody now specifically binds to the fixed protamine antibodies. Unbound molecules, proteins and antibodies are removed from the wells in a further rinse step. Then substrate is added to the wells, in particular a substrate which can be converted in a color reaction by the enzyme of the enzyme-conjugated secondary antibody, e.g. the chromogenic substrate TMB (tetramethylbenzidine). The enzyme conjugate bound to the bottom of the wells converts the per se colorless substrate into a color, e.g. blue color. The color reaction is stopped by addition of a stop solution such that the blue color is converted e.g. into yellow. The intensity of the yellow color reaction is subsequently measured spectrophoretically e.g. at 450 nm, whereby the color intensity is proportional to the concentration of anti-protamine-1 antibodies and anti-protamine-2 antibodies bound in the wells.

The questionable sample is preferably pipetted in a volume of 100 μL/well into a microtiter plate and incubated so that proteins protamine-1 and protamine-2 are bound to the surface. Residual fluid is removed from the microtiter plate by tapping, followed by addition of blocking solution (e.g. commercially available blocking solution with casein) and a further incubation step. The blocking solution serves to occupy free binding sites on the protamine-binding surface of the microtiter plate. Non-specific binding to the surface is thus prevented, the background is reduced and the sensitivity of the procedure is enhanced. The microtiter plate is then washed with washing solution (e.g. commercially available washing solution based on TRIS-buffer with Tween and 350 mM NaCl, 10-times concentrate). The washing step removes unbound and excessive components which would otherwise interfere with the ELISA assay. Monoclonal anti-protamine-IgG antibodies (e.g. commercially available from SHAL Technologies) are adjusted with buffer (e.g. commercially available sample buffer, Candor) to a concentration of 1 μg/mL (1:2100). 100 μL/well of this solution are pipetted into the microtiter plate and incubated e.g. for 90 min at 37° C.

The microtiter plate is subsequently washed with washing buffer (e.g. commercially available washing solution), e.g. three times for each 10 sec. with 200 μL/well washing buffer each.

HRP-conjugated secondary antibodies (anti-mouse-IgG, KPL) are diluted with buffer (LowCross, Candor) to a concentration of 1 μg/mL (1:1000). 100 μL/well of this solution are pipetted into the microtiter plate and incubated e.g. 60 min at 37° C. The microtiter plate is then washed, e.g. three times with each 200 μL/well washing puffer (washing buffer Tris, Candor) for 5 min each. 100 μl of TMB substrate is added to each well and incubated in the dark for 5 min, followed by addition of 100 μL/well H₂SO₄ as stop solution and measurement of the extinction (optical density, OD) of the sample at 450 nm.

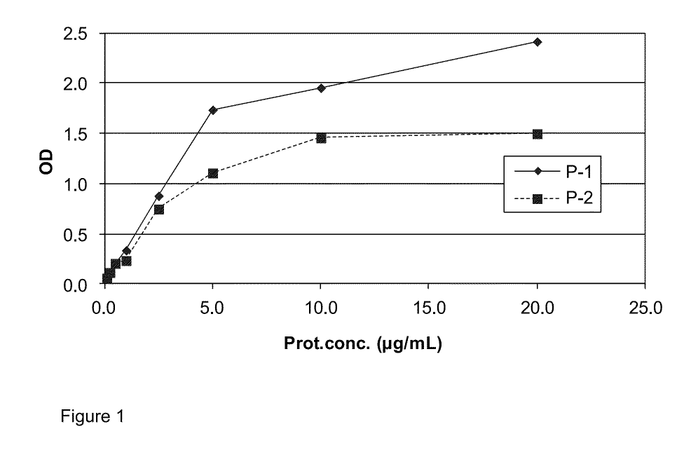

FIG. 1 shows an analysis of OD-values at protein concentrations of 0.1 μg/mL, 0.25 μg/mL, 0.5 μg/mL, 1.0 μg/mL, 2.5 μg/mL, 5.0 μg/mL, 10.0 μg/mL and 20 μg/mL of protamine-1 and protamine-2.

### 2.2. ELISA-Procedure (Competitive Procedure)

The ELISA procedure is alternatively performed in a competitive assay. The starting material is a processed questionable sample according to embodiment 1, i.e. a suspension containing decondensed sperm with a protein concentration adjusted to e.g. 0.1 μg/mL, 0.25 μg/mL, 0.5 μg/mL, 1.0 μg/mL, 2.5 μg/mL, 5.0 μg/mL, 10.0 μg/mL and/or 20 μg/mL.

For this purpose, proteins protamine-1 and protamine-2 are e.g. separately bound as recombinant proteins in a solution with defined concentration, e.g. of 0.1 μg/mL, 0.25 μg/mL, 0.5 μg/mL, 1.0 μg/mL, 2.5 μg/mL, 5.0 μg/mL, 10.0 μg/mL and/or 20 μg/mL to the surface of a microtiter plate and incubated.

Residual fluid is removed from the microtiter plate by tapping and non-specific binding sites are blocked, e.g. by incubation for 30 min at 37° C. with 300 μL/well blocking solution (Candor), followed by washing of the microtiter plate, e.g. with 200 μL/well washing buffer (washing buffer Tris, Candor) for 10 sec each.

The sample extract is mixed with a suitable amount of monoclonal anti-protamine-IgG antibodies and incubated e.g. at +4° C., whereby two separate assays are carried out for protamine-1 and protamine-2 proteins.

In addition, a standard curve can be produced in parallel using defined concentrations of protamine-1 and protamine-2. For this purpose, both recombinant proteins (P-1 and P-2) are adjusted to at least three different concentrations and the respective anti-protamine antibody is added, i.e. anti-protamine-1 antibody is added to the protamine-1 assay and anti-protamine-2 antibody is added to the protamine-2 assay.

100 μL of the sample extract-antibody mixture are pipetted into each well and incubated, e.g. 90 min at 37° C. The microtiter plate is then washed, e.g. three times with 200 μL/well washing buffer each (washing buffer Tris, Candor) for 10 sec each.

The HRP-conjugated secondary antibodies (anti-mouse IgG, KPL) are diluted with buffer (LowCross, Candor) to a defined concentration (e.g. 1 μg/mL). 100 μL are pipetted into each well and incubated for 60 min at 37° C. The microtiter plate is then rinsed, e.g. three times for 5 min each with each 200 μL/well washing buffer (washing buffer Tris, Candor).

100 μL of the TMB substrate are then pipetted into each well and the assay is incubated in the dark, followed by addition of 100 μL/well stop solution H₂SO₄ and measurement of the extinction of the sample in the microtiter plate at 450 nm.

In this competitive ELISA, the intensity of the signal is inversely proportional to the amount of the antigen (protamine-1 or protamine-2) in the sample. The higher the amount of free protamine present in the sample extract, the fewer antibodies will bind to the recombinant protamine fixed on the microtiter plate. The exact concentration of protamine-1 and protamine-2 in the respective questionable sample is then calculated using the protamine-1 and protamine-2 standard curves which are produced in parallel. Based on these values, the protamine/protein ratio can be determined which allows a statement with respect to the potential male fertility and success of in-vitro fertilization.

### 2.3. Rapid Test

The procedure of the present invention for determining the fertility of sperm, advantageously of human sperm, is alternatively performed in a rapid test on a test strip. The rapid test can be conducted by anyone without any special knowledge and without elaborate laboratory equipment at any place. The result with respect to the presence of fertility is simple and quickly evaluated.

With this rapid test, for the first time both proteins to be detected protamine-1 and protamine-2 can separately be determined qualitatively and semi-quantitatively. The evaluation includes a representation of the quantitative proportion of proteins protamine-1 to protamine-2 in relation to each other in an easy and visual way, e.g. in a color reaction in sections 107, which is immediately visible to the human eye and consequently allows a direct statement with respect to the potential male fertility.

The test strip of this invention advantageously comprises a base support 101 composed of a suitable material like e.g. nylon. Said base support 101 is square or round, preferably rectangular.

The test strip of this invention advantageously possesses a length of at least 4 cm and a width of 3 to 5 mm per zone, preferably 4 mm.

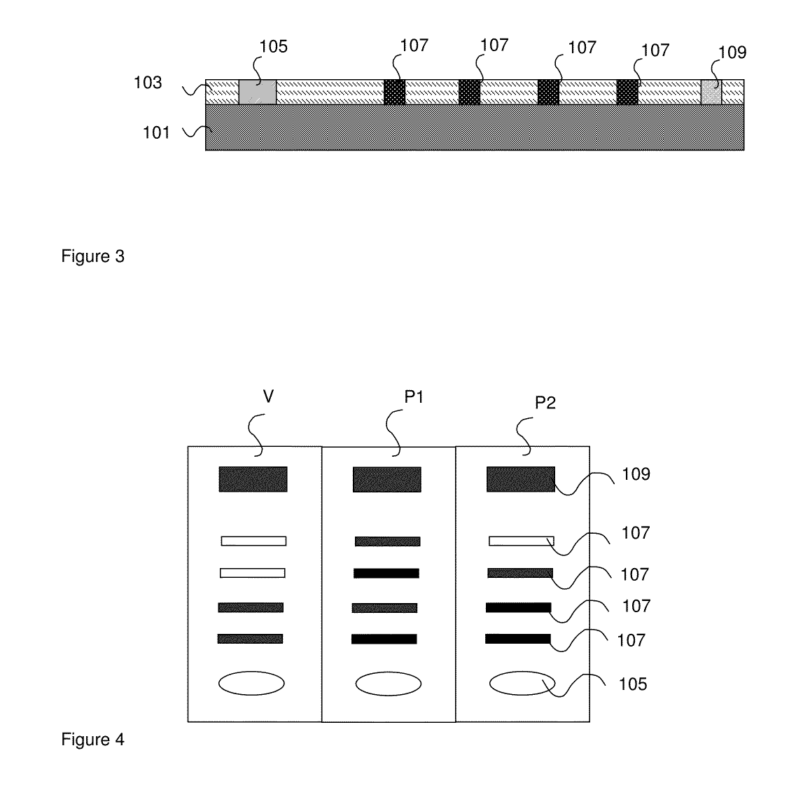

Base support 101 is furthermore coated with a suitable material, e.g. cellulose acetate or cellulose nitrate, which results the formation of application layer 103 with a few μm thickness providing a planar surface (see FIG. 3). The test strip is separated into several zones. Zone V is specifically designed for a sperm pretest in which the sperm concentration suitable to conduct said rapid test is determined. Furthermore provided are zones P1 and P2 for the protamine rapid test. Zone P1 is designed for the specific detection of antigen protamine-1 and zone P2 for the specific detection of antigen protamine-2.

Application layer 103 is provided as continuous layer extending across zones V, P1 and P2.

Each zone V, P1 and P2 is equipped with sections on the application layer 103, namely each one section 105 for sample application of a questionable sample, each at least one section 107 and each one section 109 as control section.

A preferred alternative of the test strip is equipped with an additional section 104 consisting of absorbent material, e.g. silica gel, which supports and enhances diffusion of the questionable sample across the sections. Absorbent section 104 is located at the end of the test strip distal of section 109 so that diffusion of the questionable sample from section 105 across the at least one section 107 to section 109 is supported and accelerated.

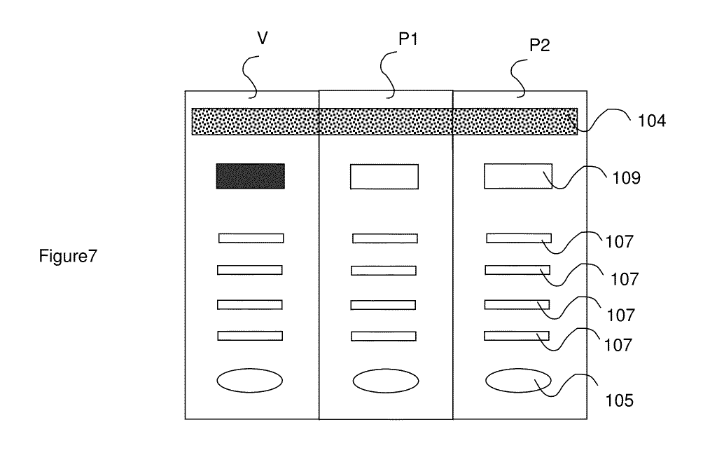

Section 104 is provided as continuous section extending across zones V, P1 and P2 (see FIG. 7), alternatively provided are separate sections 104 for each zone V, P1 and P2 (see FIGS. 12, 13 and 14).

### 2.3.1. Sperm Pretest

### Device

The sperm pretest is conducted in zone V of the test strip. This test represents a semi-quantitative pretest for determining the sperm concentration in a questionable sample, e.g. lysate of an ejaculate sample.

Based on the result of the sperm pretest, the questionable sample is either used unmodified for the subsequent protamine rapid test or a defined sperm concentration is adjusted by adding a corresponding volume of dilution buffer prior to using the questionable sample for the protamine rapid test.

In section 105, the sperm pretest contains antibodies directed against sperm surface antigens. Advantageously, section 105 contains a mixture of colloidal gold-labeled antibodies directed against sperm surface antigens and non-labeled antibodies directed against sperm surface antigens.

Upon application of the questionable sample in section 105, the sperm contained therein are bound by antibodies directed against sperm surface antigens. As a result, complexes of sperm surface antigens bound to antibodies directed against these antigens are formed which are, due to the colloidal gold labeling of the antibody, visible to the human eye as color reaction, for example as red coloring.

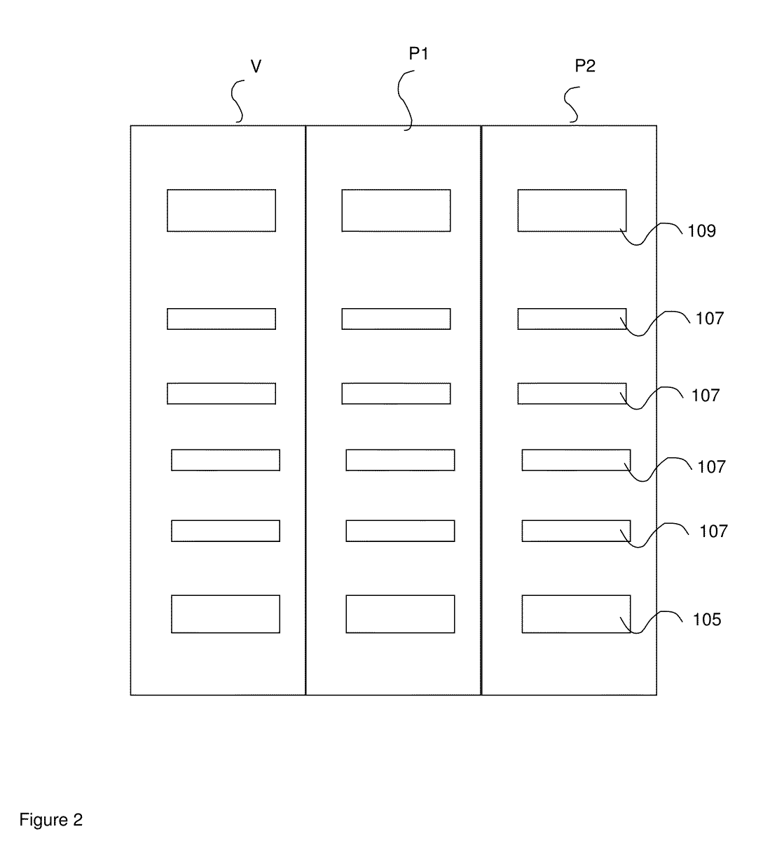

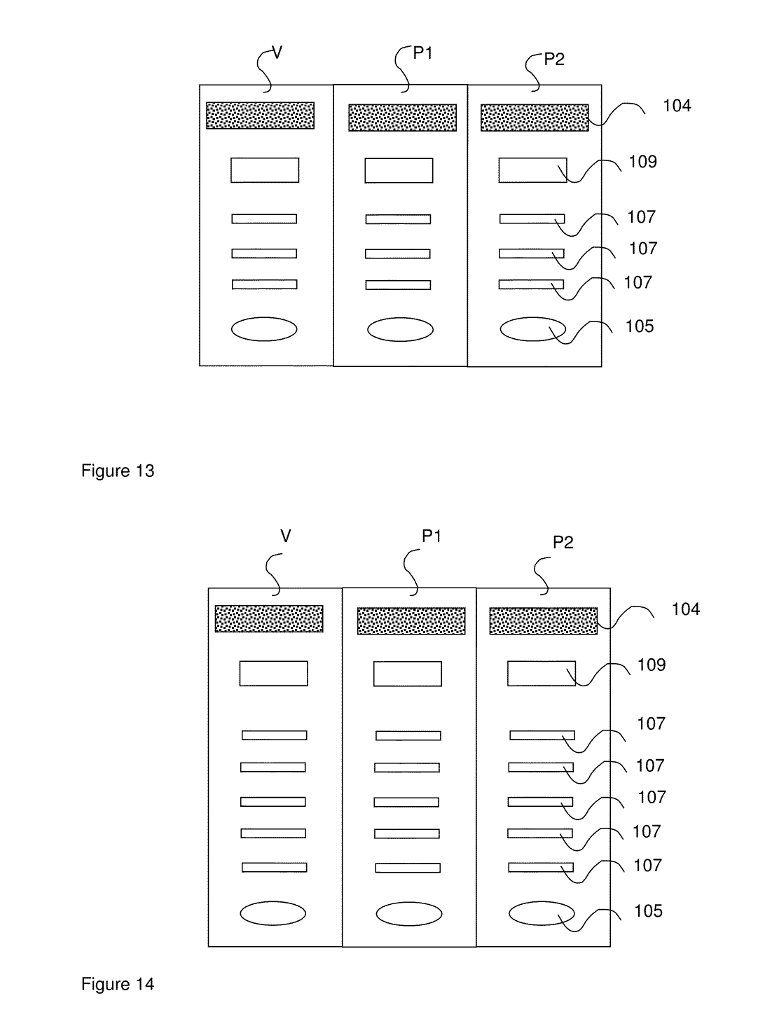

At least one section 107, advantageously a plurality of sections 107 e.g. two to ten, particularly preferred four sections 107 are arranged successively on the test strip between section 105 and control section 109 (see FIG. 2). FIG. 13 illustrates an alternative embodiment with three sections 107. FIG. 14 illustrates a further alternative embodiment with five sections 107. Sections 107 are arranged evenly spaced, the spacing alternatively may vary. Sections 107 contain unlabeled antibodies directed against complexes of sperm surface antigens bound to antibodies directed against these antigens.

Sections 107 alternatively contain different amounts of antibodies against complexes of sperm surface antigens bound to antibodies directed against these antigens which results in varying detection sensitivities.

The section 107 located most proximate to control section 109 is advantageously equipped with the highest concentration of antibodies against complexes of sperm surface antigens bound to antibodies directed against these antigens.

The amount of antibodies against complexes of sperm surface antigens bound to antibodies directed against these antigens provided in sections 107 decreases in the direction toward the section 107 which is located farthest from control section 109.

Control section 109 on zone V is equipped with unlabeled antibodies against colloid-labeled sperm surface antigens and represents a control for the validity of the test.

### Procedure in Zone V

A questionable sample prepared according to 1., e.g. an ejaculate sample is applied in a suitable volume e.g. at least 50 μL to section 105 of zone V. Alternatively, the test strip is immersed in the questionable sample such that only section 105 of zone V is completely wetted by the questionable sample.

The questionable sample, e.g. ejaculate, is advantageously incubated prior to assessment to allow for liquefaction, preferably for 30 min at room temperature.

If sperm is present in the questionable sample sperm, said sperm will bind to the colloidal gold-labeled and non-labeled antibodies against sperm-surface antigens already provided in section 105 such that labeled and non-labeled complexes between sperm-surface antigens and antibodies directed against these antigens are formed. Colloidal gold-labeled complexes induce a color reaction in section 105 which is visible to the human eye, in particular as red coloring.

The questionable sample containing colloidal gold-labeled and non-labeled complexes of sperm-surface antigens bound to antibodies directed against these antigens migrates via diffusion along the test strip in zone V and successively reaches sections 107 and control section 109.

In sections 107, the formed colloidal gold-labeled and non-labeled complexes between sperm-surface antigens and antibodies directed against these antigens come into contact with the already provided non-labeled antibodies against said formed complexes and into contact with antibodies against sperm-surface antigens. Depending on the respective sperm concentration of the sample, in sections 107 a binding of this antibody to the formed complexes now becomes visible as color reaction in either none to up to all sections 107 (see FIGS. 4 to 7). The respective number of sections 107 with visible color reaction will consequently allow a statement concerning the sperm concentration present in the questionable sample.

The presence of a visible color reaction in a different number of sections 107 of the test strip in zone V is related to the sperm concentration in the questionable sample.

If at least one but not all sections 107 in zone V exhibit a positive color reaction, the sperm concentration of the questionable sample is in an optimal range and between 0.2 millions and 200 millions of sperm/mL. This questionable sample can thus be used without further changes for the protamine rapid test in zones P1 and P2.

If none of the sections 107 in zone V shows a positive color reaction, the sperm concentration in the questionable sample is too low and the questionable sample cannot be used as such but has to be concentrated prior to usage in the assay.

If all sections 107 in zone V show a positive color reaction, the sperm concentration in the questionable sample is too high and dilution of the questionable sample is recommended to obtain the optimum range of 0.2 million sperm/mL to 200 million sperm/mL. This dilution is preferably performed with dilution buffer.

The determination of the sperm concentration in the questionable sample in zone V is advantageously completed after 10 min.

Alternatively, the sperm concentration in the questionable sample, e.g. an ejaculate sample, is determined photometrically and, if required, adjusted to the optimal sperm concentration of 0.2 million sperm/mL to 200 million sperm/mL.

### Evaluation of the Sperm Pretest

The sperm pretest is evaluated according to the following instructions:
a) Evaluation of the sperm pretest is possible if a color reaction is clearly visible in control section 109 of zone V of the sperm pretest (see FIGS. 4, 5, 6, 7, 11).
b) Evaluation of the sperm pretest is not possible if no color reaction is visible in control section 109 of zone V of the sperm pretest.
c) If none of the sections 107 in zone V shows a visible color reaction, the sperm concentration is too low to conduct the subsequent protamine rapid test.
d) If all sections 107 in zone V of the sperm pretest show a visible color reaction, the questionable sample, e.g. ejaculate, is to be diluted with dilution buffer and adjusted to an optimum sperm concentration of 0.2 million sperm/mL to 200 million sperm/mL.
e) If zone V of the sperm pretest shows a color reaction in at least one of the sections 107 but not in all sections 107, the sperm concentration is in the optimal range of 0.2 million sperm/mL to 200 million sperm/mL and the protamine rapid test can be conducted.

### 2.3.2. Protamine Rapid Test

### Device

The protamine rapid test is conducted in zones P1 and P2 of the test strip (see FIGS. 2, 3). The assay is a semi-quantitative assay for determining the protamine-1 and protamine-2 concentration in a questionable sample, e.g. the lysate of an ejaculate sample.

In said protamine rapid test, the protamine/protein ratio (ratio between the amount of protamine-1 and the amount of protamine-2) is determined on the basis of color reactions in sections 107 of zones P1 and P2. This protamine/protein ratio is a valid prognostic marker for the success of a subsequent in-vitro fertilization or intracytoplasmic sperm injection. If the protamine/protein ratio in sperm or spermatids of a questionable sample is 0.8 to 1.2 protamine-1: 0.8 to 1.2 protamine-2 or preferably equals 1:1, sperm fertility is normal and the sperm can be used for a subsequent in-vitro fertilization or intracytoplasmic sperm injection. If the ratio however deviates from this threshold range, is this indicative of reduced sperm fertility up to infertility of the sperm or spermatids in the questionable sample. This sperm is not used for a subsequent in-vitro fertilization or intracytoplasmic sperm injection.

### Zone P1

In section 105 of zone P1 of the test strip for the protamine rapid test, anti-protamine-1 antibodies are provided. Section 105 advantageously contains a mixture of colloidal gold-labeled anti-protamine-1 antibodies and non-labeled anti-protamine-1 antibodies.

Upon application of the questionable sample in section 105, the protamine-1 contained therein is bound by anti-protamine-1 antibodies which results in the formation of a complex between protamine-1 and anti-protamine-1 antibodies. This complex is visible to the human eye as color reaction due to the colloidal gold-labeling, e.g. as red coloring.

At least one section 107, advantageously several sections e.g. two to ten, particularly preferred four sections 107 are successively arranged on the test strip between section 105 and control section 109 (see FIGS. 2, 3).

The plurality of sections 107 are arranged evenly spaced, the spacing may alternatively vary. Sections 107 contain unlabeled antibodies against colloidal gold-labeled complexes between protamine-1 and anti-protamine-1 antibody.

The section 107 which is located in closest proximity to the control section 109 advantageously possesses the highest concentration of colloidal gold-labeled antibodies against said complexes between protamine-1 and anti-protamine-1 antibody.

The amount of antibodies against colloidal gold-labeled complexes between protamine-1 and anti-protamine-1 antibody in sections 107 decreases towards the direction of the section 107 which is located farthest from control section 109.

The number of sections 107 in zone P1 preferably corresponds to the number of sections 107 in zone P2.

The test strip possesses a control section 109 in zone P1 which contains unlabeled antibodies against colloidal gold-labeled anti-protamine-1 antibody and thus represents a control for the validity of the test.

### Zone P2

In section 105 of zone P2 of the test strip, the protamine rapid test contains anti-protamine-2 antibodies. Section 105 advantageously contains a mixture of colloidal gold-labeled anti-protamine-2 antibodies and non-labeled anti-protamine-2 antibodies.

Upon application of the questionable sample in section 105, the protamine-2 contained therein is bound by anti-protamine-2 antibodies which results in the formation of a complex between protamine-2 and anti-protamine-2 antibodies. This complex is visible to the human eye as color reaction due to the colloidal gold-labeling, e.g. as red coloring.

At least one section 107, advantageously several sections e.g. two to ten, particularly preferred four sections 107 are successively arranged on the test strip between section 105 and control section 109 (see FIGS. 2, 3).

The plurality of sections 107 are arranged evenly spaced, the spacing may alternatively vary. Sections 107 contain unlabeled antibodies against colloidal gold-labeled complexes between protamine-2 and anti-protamine-2 antibody.

The section 107 which is located in closest proximity to the control section 109 advantageously possesses the highest concentration of colloidal gold-labeled antibodies against said complexes between protamine-2 and anti-protamine-2 antibody.

The amount of antibodies against colloidal gold-labeled complexes of protamine-2 and anti-protamine-2 antibody in sections 107 decreases in the direction of the section 107 which is located farthest from control section 109.

The number of sections 107 in zone P2 preferably corresponds to the number of sections 107 in zone P1.

The test strip possesses a control section 109 in zone P2 which contains unlabeled antibodies against colloidal gold-labeled anti-protamine-2 antibody and thus represents a control for the validity of the test.

### Procedure P1

The questionable sample adjusted to an optimal sperm concentration of 0.2 million sperm/mL to 200 million sperm/mL, e.g. an ejaculate sample, is applied in a suitable volume e.g. at least 50 μL in section 105 in zone P1 of the test strip for the protamine rapid test.

The test strip is alternatively immersed in the questionable sample in such a way that only section 105 in zone P1 is fully wetted by the questionable sample.

In a further alternative, the test strip is immersed in such a way that only section 105 of zone P1 and section 105 of zone P2 are simultaneously and completely wetted by the questionable sample.

If protamine-1 is present in the questionable sample, said protamine-1 will bind to the colloidal gold-labeled and non-labeled antibodies against protamine-1 (anti-protamine-1 antibodies) which are provided in section 105 so that labeled and non-labeled complexes of protamine-1 and anti-protamine-1 antibodies are formed.

Said colloidal gold-labeled complexes induce a color reaction in section 105 which is visible to the human eye, in particular a red coloring.

The questionable sample with labeled and non-labeled complexes of protamine-1 and anti-protamine-1 antibodies formed migrates via diffusion along the test strip in zone P1 and successively reaches sections 107 and control section 109. In sections 107, the labeled and non-labeled complexes of protamine-1 and anti-protamine-1 antibodies which were formed come into contact with non-labeled antibodies against said formed complexes which are provided in these sections. Depending on the respective protamine-1 concentration in the sample, a binding of these antibodies to the formed complexes becomes visible as color reaction in none up to all sections 107 (see FIGS. 4, 5, 6, 7, 11), so that an estimation of the protamine-1 concentration in the sample is possible on the basis of the number of sections 107 with color reaction.

The presence of a color reaction in a varying number of sections 107 in zone P1 of the test strip is indicative of the protamine-1 concentration in the questionable sample.

The control section 109 of zone P1 is equipped with a non-labeled antibody directed against colloidal gold-labeled anti-protamine-1 antibodies. The labeling causes a color reaction upon binding which is visible to the human eye. This color reaction serves as control for correct functioning of the test strip and indicates that the sample has migrated all the way up to the end of the test strip.

A determination of the protamine-1 concentration in a questionable sample in zone P1 is advantageously completed after 10 min.

### Procedure P2

The questionable sample adjusted to an optimal sperm concentration of 0.2 million sperm/mL to 200 million sperm/mL, e.g. an ejaculate sample, is applied in a suitable volume e.g. at least 50 μL in section 105 in zone P2 of the test strip for the protamine rapid test.

The test strip is alternatively immersed in the questionable sample in such a way that only section 105 in zone P2 is fully wetted by the questionable sample.

In a further alternative, the test strip is immersed in such a way that only section 105 of zone P1 and section 105 of zone P2 are simultaneously and completely wetted by the questionable sample.

If protamine-2 is present in the questionable sample, said protamine-2 will bind to the colloidal gold-labeled and non-labeled antibodies against protamine-1 (anti-protamine-2 antibodies) which are provided in section 105 so that labeled and non-labeled complexes of protamine-2 and anti-protamine-2 antibodies are formed. Said colloidal gold-labeled complexes induce a color reaction in section 105 which is visible to the human eye, in particular a red coloring.

The questionable sample with labeled and non-labeled complexes of protamine-2 and anti-protamine-2 antibodies formed therein diffuses the test strip in zone P2 and successively reaches sections 107 and control section 109. In sections 107, the labeled and non-labeled complexes of protamine-2 and anti-protamine-2 antibodies which were formed come into contact with non-labeled antibodies against said formed complexes which are provided in these sections. Depending on the respective protamine-2 concentration in the sample, a binding of these antibodies to the formed complexes becomes visible as color reaction in none up to all sections 107 (see FIGS. 4, 5, 6, 7, 11), so that an estimation of the protamine-2 concentration in the sample is possible on the basis of the number of sections 107 with color reaction.

The presence of a color reaction in a varying number of sections 107 in zone P2 of the test strip is indicative of the protamine-2 concentration in the questionable sample.

The control section 109 of zone P2 is equipped with a non-labeled antibody directed against colloidal gold-labeled anti-protamine-2 antibodies. The labeling causes a color reaction upon binding which is visible to the human eye. This color reaction serves as control for correct functioning of the test strip and indicates that the sample has migrated all the way up to the end of the test strip.

A determination of the protamine-2 concentration in a questionable sample in zone P2 is advantageously completed after 10 min.

### Evaluation of the Protamine Rapid Test

The protamine-rapid test can be evaluated if a color reaction is clearly visible both in control section 109 of zone P1 and in control section 109 of zone P2 (see FIGS. 4, 5, 11).

Evaluation of the protamine rapid test is not possible if one or both color reactions are not clearly visible in control section 109 of zone P1 or control section 109 of zone P2 (see FIGS. 6, 7).

### 2.3.3. Evaluation of the Rapid Test

The sperm in the questionable sample is fertile if the following conditions apply:
a) The number of sections 107 in zone P1 in which a color reaction is observed has to be equal to the number of sections 107 in zone P2 in which a color reaction is observed
   and
b) the position of sections 107 in zone P1 in which a color reaction is observed has to correspond to the position of sections 107 in zone P2 in which a color reaction is observed
   meaning that that a protamine/protein ratio of 0.8 to 1.2 protamine-1: 0.8 to 1.2 protamine-2 exists, preferably a protamine/protein ratio of 1:1.

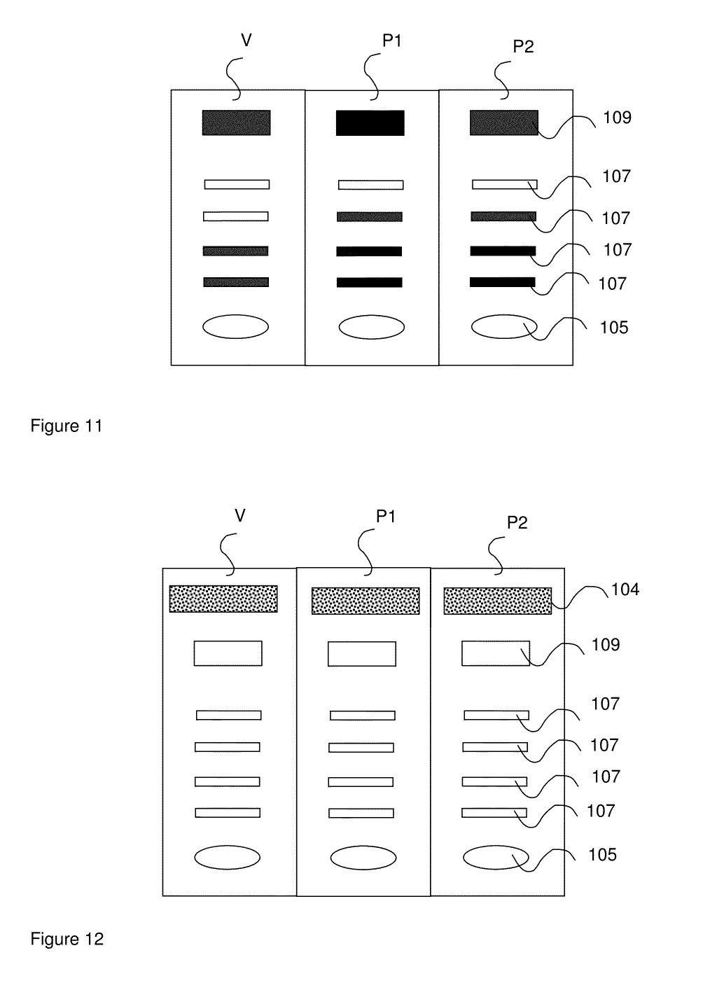

An example of a fertile questionable sample is given in FIG. 11. Following completion of the procedure, in three sections 107 in zone P1 and in three sections 107 in zone P2 a color reaction is observed, whereby the three sections 107 in zone P1 in which a color reaction is observed and the three sections 107 in zone P2 in which a color reaction is observed are located at the same position with respect to section 105 and section 109. In this example of FIG. 11 consequently as test result, a protamine/protein ratio (ratio of the amount of protamine-1 to the amount of protamine-2) of 1:1 exists.

The sperm in the questionable sample is infertile if the following conditions apply:
a) the number of sections 107 in zone P1 in which a color reaction is observed is not equal to the number of sections 107 in zone P2 in which a color reaction is observed or
b) the position of sections 107 in zone P1 in which a color reaction is observed does not correspond to the position of sections 107 in zone P2 in which a color reaction is observed meaning that that no protamine/protein ratio of 0.8 to 1.2 protamine-1: 0.8 to 1.2 protamine-2 exists, preferably no protamine/protein ratio of 1:1.

An example of an infertile questionable sample is given in FIG. 4. Following completion of the procedure, the sample shows four sections 107 in zone P1 in which a color reaction is observed and three sections 107 in zone P2 with positive color reaction.

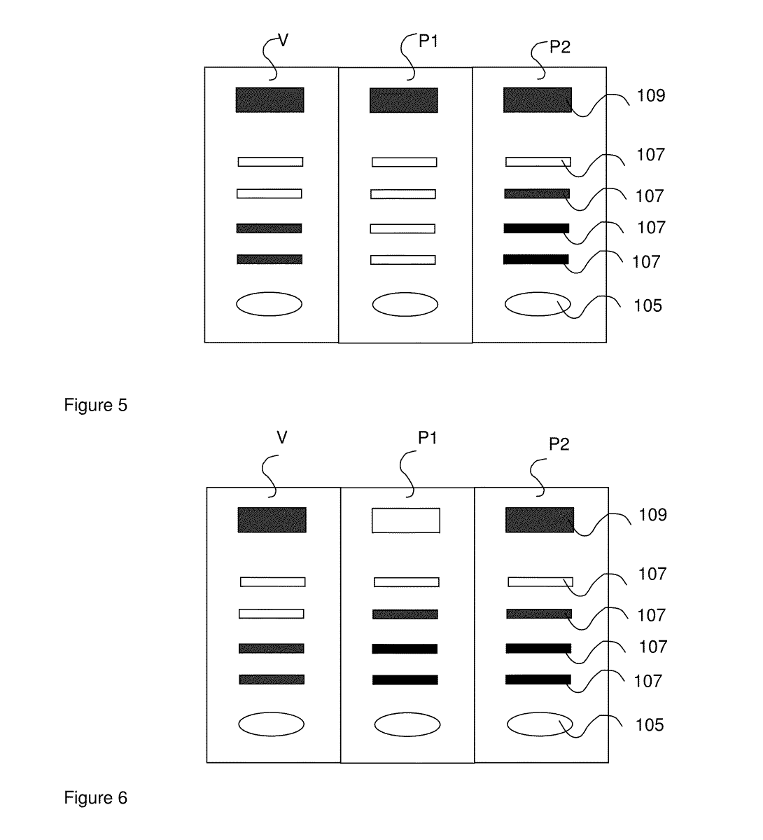

Another example for an infertile questionable sample is given in FIG. 5. Following completion of the procedure, in none of the sections 107 in zone P1 a color reaction is observed, while three sections 107 in zone P2 show a color reaction,

Figure legends

FIG. 1 shows the graphical representation of the determined optical densities (OD-values) at protein concentrations of 0.1 μg/mL, 0.25 μg/mL, 0.5 μg/mL, 1.0 μg/mL, 2.5 μg/mL, 5.0 μg/mL, 10.0 μg/mL and 20 μg/mL an protan-1 (♦ P1) and protami [symbol] -2 ( - - - - - - P2).

FIG. 2 exemplarily shows a top view of the possible arrangement of zones V, P1 and P2 and sections 105, 107 and 109 on a rectangular test strip. The zones are arranged parallel to each other and zone P1 is spatially located between the zones P2 and V.

Alternatively, zone P2 is spatially located before zones P1 and V.
Alternatively, zone V is spatially located between P1 and P2.
Alternatively, isolating separating sections are arranged between zones V, P1 and P2.

FIG. 3 depicts a cross-sectional view of an arrangement of the present invention according to FIG. 2, in particular showing the base support of the test strips 101, the application layer 103 as well as sections 105, 107 and 109 in one zone.

FIG. 4 illustrates zones V, P1 and P2 of the test strip following completion of the procedures for the sperm pretest and for the protamine rapid test. Control sections 109 in zones V, P1 and P2 each show a color reaction and a valid test result is thus obtained. In zone V, two sections 107 show a color reaction. This indicates that the sperm concentration in the questionable sample is within the optimal sperm concentration range between 0.2 million sperm/mL and 200 million sperm/mL to conduct the assay. In zone P1, four sections 107 show a color reaction. In zone P2, three sections 107 show a color reaction. This indicates that the protamine/protein ratio does not correspond to 0.8 to 1.2 protamine-1: 0.8 to 1.2 protamine-2, preferably does not corresponds to a protamine/protein ratio of 1:1. The sperm in the questionable sample is consequently infertile.

FIG. 5 illustrates zones V, P1 and P2 of the test strip following completion of the procedures for the sperm pretest and the protamine rapid test. Control sections 109 in zones V, P1 and P2 each show a positive color reaction, thus indicating a valid test result. In zone V, both sections 107 show a color reaction. This indicates that the sperm concentration in the questionable sample is within the optimal sperm concentration range of 0.2 million sperm/mL to 200 million sperm/mL to conduct the assay. In zone P1, none of the sections 107 shows a color reaction while in zone P2, three sections 107 show a color reaction. This result indicates that the protamine/protein ratio is not in a range of 0.8 to 1.2 protamine-1: 0.8 to 1.2 protamine-2 and does not correspond to the preferred protamine/protein ratio of 1:1. The sperm in the questionable sample is consequently infertile.

FIG. 6 shows zones V, P1 and P2 of the test strips following completion of the procedures for the sperm pretest and the protamine rapid test. Control sections 109 in zones V and P2 both show a color reaction. In control section 109 in zone P1, no color reaction is observed. Thus no valid test result is obtained and consequently no statement can be made with respect to sperm fertility.

FIG. 7 shows zones V, P1 and P2 of the test strip following completion of the procedures for the sperm pretest and the protamine rapid test. Only control section 109 in zone V displays a color reaction. Control sections 109 in zones P1 and P2 show no color reaction. No valid test result is obtained in this case and consequently no statement can be made with respect to the fertility of the sperm. Represented is a test strip with a continuous section 104.

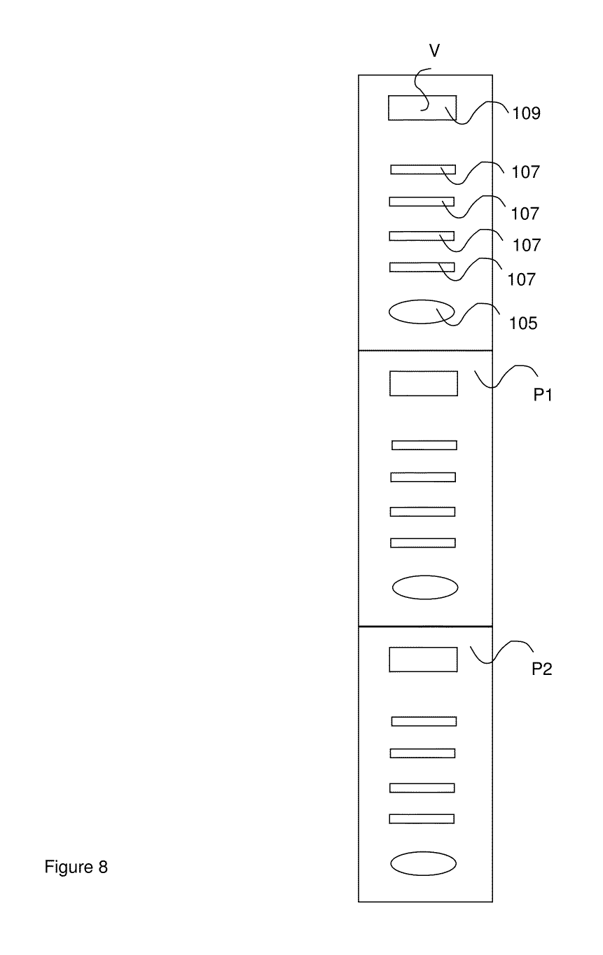

FIG. 8 illustrates an alternative possible arrangement of zones V, P1 and P2 on a rectangular test strip. The zones are arranged in successive order and zone P1 is spatially located between zones P2 and V

Alternatively, zone P2 is spatially located between zones P1 and V.

Alternatively, zone V is spatially located between zones P1 and P2.

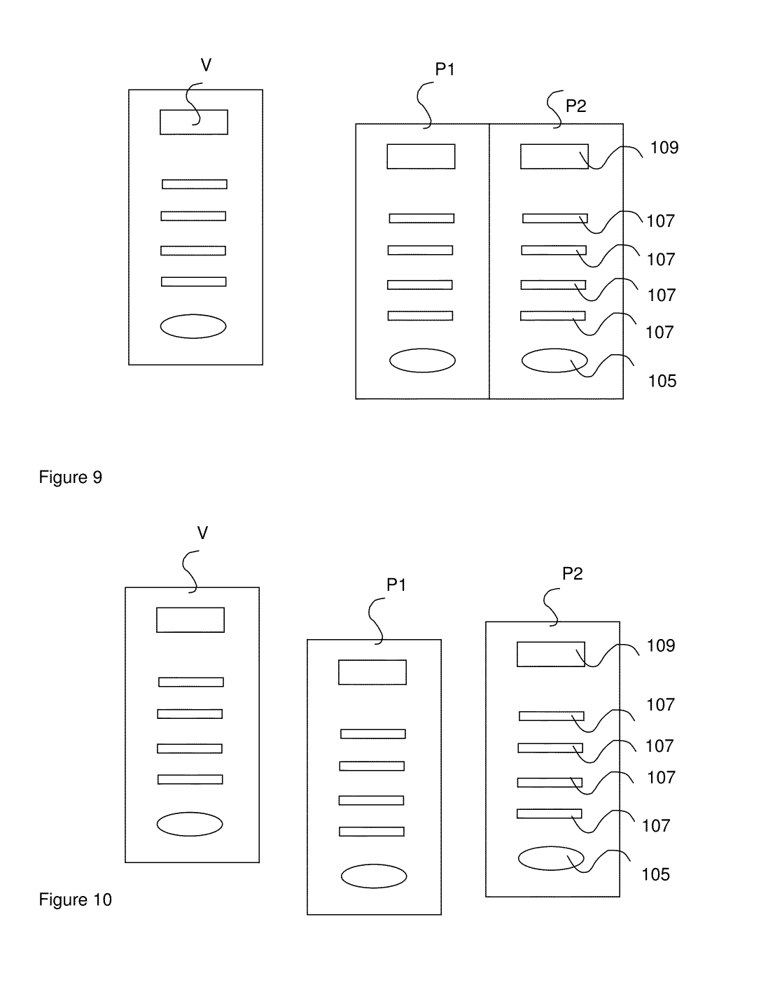

FIG. 9 illustrates a further alternative of a possible arrangement of zones V, P1 and P2. Zone V is here located on a separate test strip and zones P1 and P2 are both arranged on the same test strip.

FIG. 10 illustrates a further alternative of a possible arrangement of zones V, P1 and P2. Zones V, P1 and P2 are in this case each provided on a separate test strip.

FIG. 11 shows zones V, P1 and P2 of the test strip after completion of the procedures for the sperm pretest and the protamine rapid test. The control sections 109 in zones V, P1 and P2 each show a color reaction. A valid test result is therefore obtained. In zone V, two sections 107 show a color reaction. This indicates that the sperm concentration in the questionable sample lies within the sperm concentration range of 0.2 million sperm/mL to 200 million sperm/mL which is optimal to perform the assay. In zone P1, three sections 107 show a color reaction. In zone P2, again three sections 107 show a color reaction. This indicates that a protamine/protein ratio of 0.8 to 1.2 protamine-1: 0.8 to 1.2 protamine-2 exists, advantageously a protamine/protein ratio of 1:1. The sperm in the questionable sample is thus fertile.

FIG. 12 shows zones V, P1 and P2 of the test strip. Represented is a test strip with separate sections 104 for each zone (V, P1 and P2). Each zone (V, P1 and P2) possesses four sections 107.

FIG. 13 shows zones V, P1 and P2 of the test strip. Represented is a test strip with separate sections 104 for each zone (V, P1 and P2). Each zone (V, P1 and P2) possesses three sections 107. FIG. 14 shows zones V, P1 and P2 of the test strip. Represented is a test strip with separate sections 104 for each zone (V, P1 and P2). Each zone (V, P1 and P2) possesses five sections 107.

### REFERENCE LIST

V Zone V for the sperm pretest

P1 Zone P1 for the detection of protamine-1

P2 Zone P2 for the detection of protamine-2

101 Base support of the test strips

103 Application layer

104 Absorbent section

105 Section for sample application

107 Section for reacting antigen with antibody

109 Control section

1) A procedure for determining the fertility of male mammals and humans, wherein the concentration of protamine-1 and protamine-2 and the ratio between protamine-1 and protamine-2, the protamine/protein ratio, is determined in vitro in a questionable sample from an individual to be examined.

2) The procedure according to claim 1, wherein the concentration of protamine-1 and protamine-2 is determined by immunological procedures.

3) The procedure according to claim 1, wherein the questionable sample from an individual to be examined is ejaculate, testicular tissue or epididymis tissue.

4) The procedure according to claim 1, wherein in the immunological procedure an antibody specific for protamine-1 (anti-protamine-1 antibody) and an antibody specific for protamine-2 (anti-protamine-2 antibody) are utilized, whereby binding of protamine-1 to the anti-protamine-1 antibody and binding of protamine-2 to the anti-protamine-2 antibody occurs and this binding is made visible through color reactions.

5) The procedure according to claim 1, wherein the concentration of protamine-1 and protamine-2 is determined using an enzyme-linked immunosorbent assay (ELISA).

6) The procedure according to claim 1, wherein the concentration of protamine-1 and protamine-2 and the protamine/protein ratio is determined using a rapid test on a test strip.

7) The procedure according to claim 6, wherein the rapid test on the test strip comprises a sperm pretest and a protamine rapid test.

8) The procedure according to claim 6, wherein the sperm concentration is determined in the sperm pretest, whereby antibodies against sperm surface antigens are utilized and the binding of these antibodies to sperm-surface antigens is made visible through a color reaction.

9) The procedure according to claim 6, wherein the concentration of protamine-1 and protamine-2 is determined in the protamine rapid test whereby the concentration of protamine-1 is determined using anti-protamine-1 antibodies and the concentration of protamine-2 is determined using anti-protamine-2 antibodies and these bindings are made visible through color reactions.

10) The procedure according to claim 6, wherein the antibodies against sperm surface antigens utilized for the sperm pretest represent a mixture of colloidal gold-labeled and non-labeled antibodies.

11) The procedure according to claim 6, wherein anti-protamine-1 antibodies and the anti-protamine-2 antibodies used in the protamine rapid test are a mixture of colloidal gold-labeled antibodies and non-labeled antibodies.

12) A test strip to conduct a procedure according to claim 6, wherein said test strip comprises a base support and an application layer, whereby on said application layer a zone V is provided for conducting the sperm pretest to determine the concentration of the sperm, furthermore a zone P1 to determine the concentration of protamine-1 and a zone P2 to determine the concentration of protamine-2.

13) A test strip according to claim 12, wherein in zones V, P1 and P2 sections are provided, namely each one section for application of a questionable sample, each at least one section and each one section as control section.

14) A test strip according to claim 12, wherein the sperm pretest to determine the sperm concentration is conducted in zone V, whereby section contains antibodies against sperm surface antigens and the at least one section contains antibodies against complexes between sperm surface antigens and antibodies directed said antigens and said bindings are made visible through color reactions.

15) A test strip according to claim 12, wherein in zone P1 the concentration of protamine-1 is determined, whereby section is equipped with anti-protamine-1 antibodies and the at least one section is equipped with antibodies which bind to the complex between protamine-1 and anti-protamine-1 antibodies and said bindings are made visible through color reactions.

16) A test strip according to claim 12, wherein in zone P2 the concentration of protamine-1 is determined, whereby section is equipped with anti-protamine-2 antibodies and the at least one section is equipped with antibodies which bind to the complex between protamine-2 and anti-protamine-2 antibodies and said bindings are made visible through color reactions.

17) A test strip according to claim 12, wherein zones V, P1 and P2 are arranged in parallel to each other.

18) The utilization of a test strip according to claim 12 for determining the concentration of protamine-1 and protamine-2 and the ratio between protamine-1 and protamine-2 as protamine/protein ratio in a questionable sample from an individual to be examined in order to assess the fertility of the sperm in vitro.
